(19) United States
(12) Patent Application Publication
(10) Pub. No.: US 2009/0043378 A1
(43) Pub. Date: Feb. 12, 2009

(54) Biocompatible Polymer System for Extended Drug Release

(75) Inventors: Peiwen Cheng, Santa Rosa, CA (US); Mingfei Chen, Santa Rosa, CA (US); Kishore Udipi, Santa Rosa, CA (US)

Correspondence Address: MEDTRONIC VASCULAR, INC.;IP LEGAL DEPARTMENT, 3576 UNOCAL PLACE, SANTA ROSA, CA, 95403, US

(73) Assignee: Medtronic Vascular, Inc., Santa Rosa, CA (US)

(21) Appl. No.: 11/861,822

(22) Filed: Sep. 26, 2007

Related U.S. Application Data
(60) Provisional application No. 60/955,286, filed on Aug. 10, 2007.

(51) Int. Cl. A61F 2/82; A61K 31/4353; A61P 9/00

(52) U.S. Cl. 623/1.42; 523/113; 514/291; 424/423; 623/1.46

(57) ABSTRACT

A self-orienting biocompatible polymer system incorporating a hydrophilic surface and a hydrophobic core are disclosed. The hydrophilic surface aids in biocompatibility while the hydrophobic core allows the polymer system to accommodate a hydrophobic drug. Medical devices coated with the polymer system are also disclosed.

### RELATED APPLICATIONS

This application claims the benefit of U.S. Provisional Patent Application No. 60/955,286, filed Aug. 10, 2007.

### FIELD OF THE INVENTION

Biocompatible coatings for medical devices are described herein. More specifically, polymer coatings designed to be more biocompatible than previous coatings and allow for more sustained delivery of hydrophobic bioactive agents are described. The polymer system described herein comprises a hydrophilic surface and a hydrophobic core.

### BACKGROUND OF THE INVENTION

Medical devices are constantly evolving into more complex, helpful and useful products. Medical devices can be simple ex vivo devices such as adhesive bandages, canes, walkers and contact lenses, or complex implantable devices including pacemakers, heart valves, vascular stents, catheters and vascular grafts. Implantable devices, among other things, must be biocompatible as to alleviate the adverse physiological reactions of a rejected implant and its recipient.

Introduction of the drug-eluting stent (DES) in coronary intervention has made a significant difference in lowering restenosis rates from about 30% to the single digits. Restenosis refers to the arterial renarrowing resulting from the body's response to the vascular injury that occurs during an interventional procedure. Neointimal hyperplasia resulting from proliferation and migration of smooth muscle cells and the production of extracellular matrix are responsible for the lumen loss.

Development of drug-eluting stents relies on polymers to provide a platform for the delivery of drugs. Sustained local delivery of the drug from the stent at a controlled rate is critical to derive full benefit and, in this respect, polymer architecture assumes a vital role. Polymers play a critical role in local drug delivery from the stent scaffold and, to date, attempts to deliver drug without polymer have not proven successful. However, synthetic polymer coatings have been postulated to elicit an inflammatory and/or thrombotic response in the arterial wall.

The majority of first generation DES coatings are based on hydrophobic polymers which retain and release drug in a controlled fashion. It is believed that their hydrophobic profile which results in a lack of biocompatibility and ultimately contributes to adverse events in vivo, such as delayed healing and late stent thrombosis. Bioabsorbable polymers are often an alternative to biostable polymers for drug-eluting stent coatings. These polymers degrade temporally, leaving behind only a bare metal stent. However, the biocompatibility of these polymers, specifically in a vascular setting, depends to a large extent on degradation kinetics. Faster degrading glycolide-based polymers can enhance local acidity rapidly to elicit a strong inflammatory response. Conversely, those that are considered safer, such as polylactides, need years to degrade. Furthermore, degrading polymers can generate fragments that potentially lead to emboli. Clearly, bioabsorbable polymers for stent coatings are not without challenges and improved polymers are needed.

### SUMMARY OF THE INVENTION

Generally, provided herein is a polymer system that blends a homopolymer, a copolymer and a terpolymer to create a self-orienting, blended polymer system that exhibits a hydrophobic core for accommodating hydrophobic drugs and a hydrophilic surface that increases the polymer system's biocompatibility. The polymer system can be coated onto vascular stents and sustain delivery of a hydrophobic drug(s) for several months. The polymer system is robust and will not deteriorate, crack, or delaminate.

One embodiment is a polymer system for coating medical devices comprising a polymer blend wherein the polymer blend forms a self-orienting polymer coating having an outer surface, and wherein the polymer coating has hydrophilic groups oriented towards said outer surface. In one embodiment, the polymer blend comprises at least one of a homopolymer, a copolymer or a terpolymer.

In another embodiment, the self-orienting polymer coating is biocompatible. In another embodiment, the outer surface has a water contact angle of <950. In one embodiment, the copolymer comprises alkyl methacrylate monomers, vinyl acetate monomers or combinations thereof. In another embodiment, the terpolymer comprises alkyl methacrylate monomers, vinyl pyrrolidone monomers, vinyl acetate monomers or combinations thereof. In another embodiment, the homopolymer comprises vinyl pyrrolidone monomers.

In one embodiment, the self-orienting polymer coating is capable of controlled release of a hydrophobic drug. In one embodiment, the hydrophobic drug is selected from the group consisting of anti-proliferatives, estrogens, chaperone inhibitors, protease inhibitors, protein-tyrosine kinase inhibitors, leptomycin B, peroxisome proliferator-activated receptor gamma ligands (PPARγ), hypothemycin, nitric oxide, bisphosphonates, epidermal growth factor inhibitors, antibodies, proteasome inhibitors, antibiotics, anti-inflammatories, anti-sense nucleotides and transforming nucleic acids. In one embodiment, the drug comprises at least one compound selected from the group consisting of sirolimus (rapamycin) and its analogs, tacrolimus (FK506), everolimus (certican), temsirolimus (CCI-779), zotarolimus (ABT-578), paclitaxel and its analogs.

In another embodiment, the coating's outer surface has a Hilderbrand solubility parameter of about 15 to about 20. In another embodiment, the polyer system comprises a ratio of terpolymer to copolymer to homopolymer, wherein said ratio is from about 40:40:20 to about 88:10:2.

In one embodiment, a biocompatible medical device is described which comprises a substrate having a coating on a surface, wherein the coating comprises a self-orienting polymer system. Further, the polymer system comprises an outer surface. Further, the polymer system has hydrophilic groups oriented towards said outer surface. In one embodiment, the polymer system comprises at least one of a homopolymer, a copolymer, or a terpolymer.

In another embodiment, the medical device is implantable and is selected from the group consisting of heart valves, stents, pacemaker leads and combinations thereof.

In one embodiment, the self-orienting polymer coating is capable of controlled release of a hydrophobic drug. The hydrophobic drug can comprise at least one compound selected from the group consisting of sirolimus (rapamycin) and its analogs, tacrolimus (FK506), everolimus (certican), temsirolimus (CCI-779), zotarolimus (ABT-578), paclitaxel and its analogs.

In one embodiment, the copolymer comprises alkyl methacrylate monomers, vinyl acetate monomers, or combinations thereof. In another embodiment, the terpolymer comprises alkyll methacrylate monomers, vinyl pyrrolidone monomers, vinyl acetate monomers, or combinations thereof. In another embodiment, the homopolymer comprises vinyl pyrrolidone monomers.

In one embodiment, the outer surface has a water contact angle of <95°. In another embodiment, the outer surface has a Hilderbrand solubility parameter of about 15 to about 20. In another embodiment, the polymer system comprises a ratio of terpolymer to copolymer to homopolymer, wherein said ratio is from about 40:40:20 to about 88:10:2.

In one embodiment, a biocompatible implantable stent is described, wherein the stent comprises a self-orienting polymer system coating comprising polyvinylpyrrolidone, alkyl methacrylate, vinyl acetate, and vinylpyrrolidone. The polymer system further comprises a hydrophilic surface and a hydrophobic core. The hydrophobic core comprises a hydrophobic drug and can provide controlled release. In one embodiment, the drug comprises zotarolimus.

### BRIEF DESCRIPTION OF THE DRAWINGS

FIG. 1 depicts glass transition temperatures for the component polymers and polymer blends.

FIG. 2 depicts additional determinations of glass transition temperatures for different polymers and polymer blends.

FIG. 3 depicts a visual representation of how the contact angle of a surface is measured.

FIG. 4 depicts zotarolimus drug elution profiles for stents coated with the polymers and polymer blends.

FIG. 5 depicts the chemical structures for exemplary polymers.

FIG. 6 depicts the surface characteristics of a polymer coated stent. FIG. 6A depicts a Raman map of the surface of a polymer coated stent; FIG. 6B depict the spectra acquired from the regions selected in FIG. 6A; and FIG. 6C depicts an overlay of the three spectra from FIG. 6B.

FIG. 7 depicts attenuated total reflectance spectra for C10, C19, PVP, and C10/C19/PVP.

FIG. 8 depicts the attenuated total reflectance spectra of FIG. 7 overlaid and expanded in the carbonyl region (1800-1600 cm⁻¹).

FIG. 9A & B depict the relative adhesion of monocytes to the C10, C19 and C10/C19/PVP polymers/polymer blends (FIG. 9A) and fluorescently labeled samples of the C10, C19 and C10/C19/PVP polymers/polymer blends (FIG. 9B); FIGS. 9C & D depict the relative adhesion of monocytes to the C10/C19/PVP polymer blend in comparison to other commonly used polymers (FIG. 9C) and fluorescently labeled samples of the C10/C19/PVP polymer blend in comparison to other commonly used polymers (FIG. 9D).

### DEFINITION OF TERMS

It may be helpful to set forth definitions of certain terms that will be used hereinafter:

Animal: As used herein, “animal” shall include mammals, fish, reptiles and birds. Mammals include, but are not limited to, primates, including humans, dogs, cats, goats, sheep, rabbits, pigs, horses and cows.

Biocompatible: As used herein, “biocompatible” shall mean any material that does not cause injury or death to the animal or induce an adverse reaction in an animal when placed in intimate contact with the animal's tissues. Adverse reactions include inflammation, infection, fibrotic tissue formation, cell death, or thrombosis.

Controlled Release: As used herein, “controlled release” refers to the release of a bioactive compound from a medical device surface at a predetermined rate. Controlled release implies that the bioactive compound does not come off the medical device surface sporadically in an unpredictable fashion and does not “burst” off of the device upon contact with a biological environment (also referred to herein a first order kinetics) unless specifically intended to do so. However, the term “controlled release” as used herein does not preclude a “burst phenomenon” associated with deployment. In some embodiments, an initial burst of drug may be desirable followed by a more gradual release thereafter. The release rate may be steady state (commonly referred to as “timed release” or zero order kinetics), that is the drug is released in even amounts over a predetermined time (with or without an initial burst phase) or may be a gradient release. A gradient release implies that the concentration of drug released from the device surface changes over time.

Copolymer: As used herein “copolymer” shall mean a polymer being composed of two different monomers.

Drug(s): As used herein, “drug” shall include any compound or bioactive agent having a therapeutic effect in an animal. Exemplary, non limiting examples include anti-proliferatives including, but not limited to, macrolide antibiotics including FKBP-12 binding compounds, estrogens, chaperone inhibitors, protease inhibitors, protein-tyrosine kinase inhibitors, leptomycin B, peroxisome proliferator-activated receptor gamma ligands (PPARγ), hypothemycin, nitric oxide, bisphosphonates, epidermal growth factor inhibitors, antibodies, proteasome inhibitors, antibiotics, anti-inflammatories, anti-sense nucleotides and transforming nucleic acids. Drugs can also refer to bioactive agents including anti-proliferative compounds, cytostatic compounds, cytotoxic compounds, anti-inflammatory compounds, chemotherapeutic agents, analgesics, antibiotics, protease inhibitors, statins, nucleic acids, polypeptides, growth factors and delivery vectors including recombinant micro-organisms, liposomes, and the like.

Exemplary FKBP-12 binding agents include sirolimus (rapamycin), tacrolimus (FK506), everolimus (certican or RAD-001), temsirolimus (CCI-779 or amorphous rapamycin 42-ester with 3-hydroxy-2-(hydroxymethyl)-2-methylpropionic acid as disclosed in U.S. patent application Ser. No. 10/930,487) and zotarolimus (ABT-578; see U.S. Pat. Nos. 6,015,815 and 6,329,386). Additionally, other rapamycin hydroxyesters as disclosed in U.S. Pat. No. 5,362,718 may be used in combination with the polymers described herein.

Ductility: As used herein, “ductility,” or “ductile” refers to polymer's resistance to fracture or cracking when folded, stressed or strained at operating temperatures. When used in reference to the polymer coating compositions described herein, the normal operating temperature for the coating will be between room temperature and body temperature or approximately between 15° C. and 40° C. In one embodiment, ductility is measured at or around body temperature. Polymer durability in a defined environment is often a function of its elasticity/ductility.

Glass Transition Temperature: As used herein, “glass transition temperature” or “T_{g}” is the temperature at which an amorphous polymer becomes hard and brittle like glass. At temperatures above its T_{g} a polymer is elastic or rubbery; at temperatures below its T_{g} the polymer is hard and brittle like glass. T_{g} may be predictive of elasticity/ductility.

Homopolymer: As used herein, “homopolymer” shall mean a polymer being composed of a single monomer.

Hydrophilic: As used herein, “hydrophilic” refers to a molecule or substance's affinity towards water. In reference to a drug, the term “hydrophilic” refers to a drug that has a solubility in water of more than 200 micrograms per milliliter. In reference to a polymer or polymer blend, “hydrophilic” also refers to the surface's ability to form intermolecular interactions with surrounding aqueous environments.

Hydrophobic: As used herein, “hydrophobic” refers to molecule or substance's repulsion towards water. In reference to a drug, the term “hydrophobic” refers to a drug that has a solubility in water of less than 200 micrograms per milliliter. In reference to a polymer or polymer blend, “hydrophobic” refers to the surface's inability to form intermolecular interactions with surrounding aqueous environments.

Polymer System: As used herein, “polymer system” refers to the combination of polymers described herein. The polymer system described herein has areas of both hydrophobicity and areas of hydrophilicity. As is the case herein, the polymer system self-orients in such a way that the surface of the polymer system is substantially hydrophilic and the core of the polymer system is substantially hydrophobic.

Self-orienting: As used herein, “self-orienting” shall refer to the process whereby the polymer system orients to a configuration of hydrophilic surface and hydrophobic core from a random configuration following its application onto a stent.

Terpolymers: As used herein “terpolymer” shall mean a polymer being composed of three different monomers.

Units of Measure: As used herein, solubility parameters for polymers and solvents will be expressed in δ as originally defined by Hildebrand and Hansen. δ is a thermodynamic unit expressed in J^{1/2}/cm^{3/2}. However, the reader is cautioned that beginning in 1984 a new value for δ has been adopted and designated δ(SI) and expressed in MPa^{1/2}. To convert between δ (J^{1/2}/cm^{3/2}) and δ(SI) (MPa^{1/2}) multiply δ by 2.0045 or divide δ(SI) by 0.488.

### DETAILED DESCRIPTION OF THE INVENTION

A polymer system for coating and forming implantable medical devices is described herein. The polymer system is self-orienting upon formation resulting in a hydrophilic polymer-air interface (outer surface) and a hydrophobic core. The self-orienting results in the polymer systems hydrophilic groups toward the surface and the hydrophobic groups toward the core of the polymer. The polymer system self-orients to form an outer surface, which is hydrophilic, and in an inner core which is hydrophobic.

The polymer system is comprised of a blend of polymers. The polymers may comprise hydrophilic, hydrophobic, and amphiphilic monomers and combinations thereof. In one embodiment, the polymers of the system comprise a homopolymer, a copolymer and a terpolymer.

The homopolymer comprises a hydrophilic polymer constructed of a hydrophilic monomer selected from the group consisting of poly(vinylpyrrolidone) and poly(hydroxylalkyl methacrylate).

The copolymer comprises a polymer constructed of hydrophilic monomers selected from the group consisting of vinyl acetate, vinylpyrrolidone and hydroxyalkyl methacrylate and hydrophobic monomers selected from the group consisting of alkyl methacrylates including methyl, ethyl, propyl, butyl, hexyl, octyl, dodecyl, and lauryl methacrylate and alkyl acrylates including methyl, ethyl, propyl, butyl, hexyl, octyl, dodecyl, and lauryl acrylate.

The terpolymer comprises a polymer constructed of hydrophilic monomers selected from the group consisting of vinyl acetate and poly(vinylpyrrolidone), and hydrophobic monomers selected from the group consisting of alkyl methacrylates including methyl, ethyl, propyl, butyl, hexyl, octyl, dodecyl, and lauryl methacrylate and alkyl acrylates including methyl, ethyl, propyl, butyl, hexyl, octyl, dodecyl, and lauryl acrylate.

In another embodiment the polymer system is made form three polymers, a terpolymer, a copolymer and a homopolymer. In one such embodiment the terpolymer has the lowest glass transition temperature (T_{g}), the copolymer has an intermediate T_{g} and the homopolymer has the highest T_{g}. In one embodiment the ratio of terpolymer to copolymer to homopolymer is about 40:40:20 to about 88:10:2. In another embodiment, the ratio is about 50:35:15 to about 75:20:5. In a preferred embodiment the ratio is approximately 63:27:10. The preferred embodiment, comprises a terpolymer having a T_{g} in the range of about 5° C. to about 25° C., a copolymer having a T_{g} in the range of about 25° C. to about 40° C. and a homopolymer having a T_{g} in the range of about 170° C. to about 180° C. More specifically, the polymer system comprises a terpolymer (C19) comprising the monomer subunits n-hexyl methacrylate, N-vinylpyrrolidone and vinyl acetate having a T_{g} of about 10° C. to about 20° C., a copolymer (C10) comprising the monomer subunits n-butyl methacrylacte and vinyl acetate having a T_{g} of about 30° C. to about 35° C. and a homopolymer comprising polyvinylpyrrolidone having a T_{g} of about 174° C.

In one embodiment, an exemplary polymer comprises about 63% of C19, about 27% of C10 and about 10% of polyvinyl pyrrolidone (PVP). This exemplary polymer is referred to as C10/C19/PVP. Polymers are synthesized by radical initiated solution polymerization and their physical properties have been characterized. The C10 polymer is comprised of hydrophobic n-butyl methacrylate to provide adequate hydrophobicity to accommodate zotarolimus and a small amount of vinyl acetate. The C19 polymer is soft relative to the C10 polymer and is synthesized from a mixture of hydrophobic n-hexyl methacrylate and hydrophilic N-vinyl pyrrolidone and vinyl acetate monomers to provide enhanced biocompatibility. Polyvinyl pyrrolidone (PVP) is a medical grade hydrophilic polymer.

To create a balance of hydrophilic and hydrophobic properties in single polymer architecture and achieve a combination of desired drug elution profile and biocompatibility is challenging. Often times polymers with complimentary properties are blended. However, the polymers thermodynamic properties have to be considered. Thermodynamic properties can lead to phase separation unless the polymers are designed in such a way to create a compatible blend. The C19 polymer has adequate hydrophilic units (N-vinyl pyrrolidone and vinyl acetate) to offer hydrophilicity and the n-hexyl methacrylate units having long carbonaceous hydrophobic side chains contribute to make the polymer compatible with the C10 polymer. The C10 polymer, on the other hand, is predominantly comprised of the hydrophobic n-butyl methacrylate units with a few vinyl acetate units dispersed along the polymer backbone. Similarities between the n-hexyl and n-butyl methacrylate units and the common vinyl acetate monomer in the two polymers make them compatible. Polyvinyl pyrrolidone would not be expected to be compatible with the C10 and C19 polymers. Morphological examination of the C10/C19/PVP blend and thermal transition data demonstrates that PVP is finely dispersed in the binary blend. The C19 polymer with both hydrophilic and hydrophobic units acts like a polymeric surfactant analogous to a surfactant action in oil-water mixture.

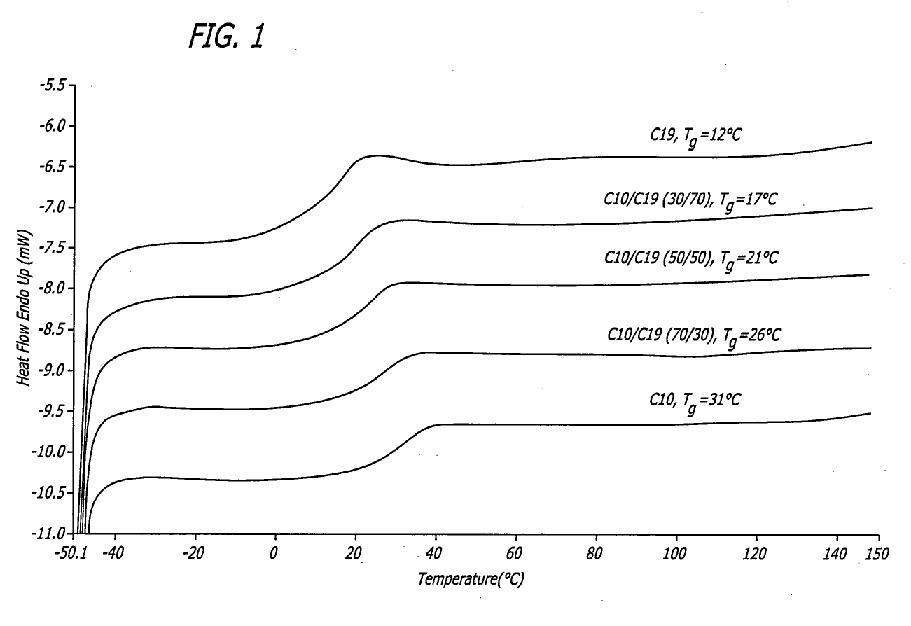

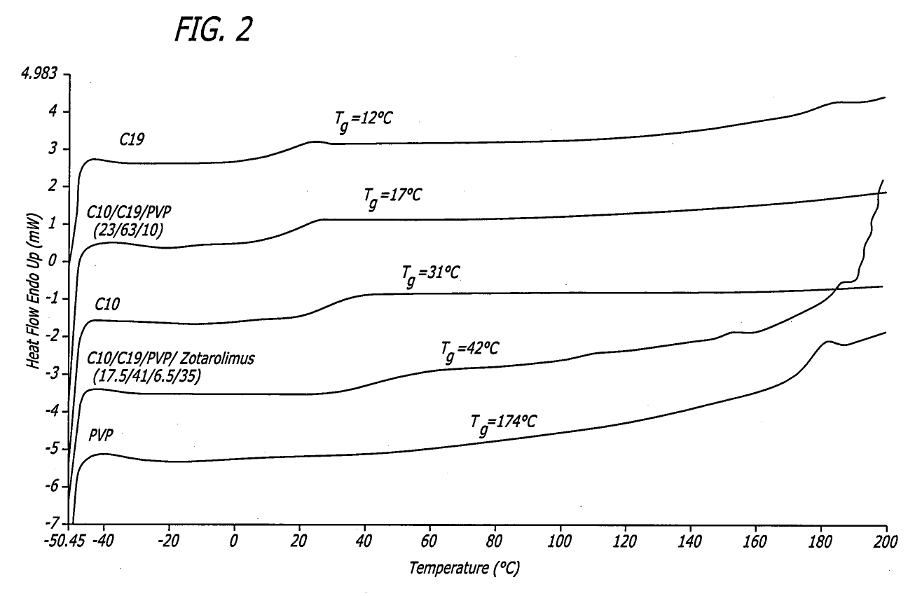

The C10 and C19 polymers can be blended in various ratios; the T_{g}s of some blends are shown in FIG. 1. Thermal analysis is a convenient and easy method to determine polymer compatibility. Unlike low molecular weight compounds, most polymer blends are thermodynamically incompatible and tend to phase separate unless they are very similar structurally or show strong interactions such as hydrogen bonding. Incompatible blends of two polymers exhibit two T_{g}s intrinsic to the two component polymers. If the two polymers are structurally very similar, like in the C10 and C19 polymers, they would be close to miscibility limits and would exhibit a single T_{g} depending on the ratio of the two in the blend, as seen for the C10/C19 blends (FIG. 1). The two polymers are miscible because both are based to a large degree on similar methacrylate monomers, n-butyl methacrylate in the C10 polymer and n-hexyl methacrylate in the C19 polymer. Incorporation of N-vinyl pyrrolidone in the C19 polymer does not affect its compatibility with the C10 polymer. Furthermore, a small amount (about 10% or less) of a third polymer, PVP, does not alter the overall compatibility of the polymer blend (FIG. 2).

The polymers and polymer systems described herein are developed to coat implantable medical devices such vascular stents, vascular stent grafts, urethral stents, bile duct stents, catheters, inflation catheters, injection catheters, guide wires, pacemaker leads, ventricular assist devices, and prosthetic heart valves. Devices such as these are generally subjected to flexion strain and stress during implantation, application or both. Providing flexible medical devices such as stents with stable biocompatible polymer coatings is especially difficult.

Biocompatibility is an important property of a drug eluting stent (DES) implant and due consideration was given to this property in designing the polymer system. This was achieved primarily by imparting a hydrophilic character into this polymer system. A polymer, when implanted in an animal, is subject to hostile foreign body response. Such a response largely depends on the polymer structure and purity. Presence of residual monomer or solvent would elicit inflammatory responses. Ideally, it would be desirable to coat the stent with a bio-friendly hydrophilic polymer. However, the coating has to also accommodate a hydrophobic drug. Hence, the polymer system was designed by blending hydrophilic and hydrophobic polymers. Long-chain methacrylate esters constitute the hydrophobic components and polar N-vinyl pyrrolidone and vinyl acetate form the hydrophilic components of the polymers. Incorporation of N-vinyl pyrrolidone in the C19 polymer increases the hydrophilicity as observed from the sessile drop contact angle measurements (Table 2).

Also, the monomers selected to synthesize these polymers should be of proven biocompatibility. Thus monomers such as vinyl acetate, n-butyl methacrylate and N-vinyl pyrrolidone have been employed successfully in commercial medical implants. The n-hexyl methacrylate monomer is only a higher homologue of n-butyl methacrylate and is expected to behave in a manner analogous to n-butyl methacrylate. It is also important that the polymers are free of residual monomers. Hence, the polymers were purified through multiple precipitations and the purity confirmed by nuclear magnetic resonance (NMR) spectroscopy and gas chromatography (GC) as a part of the synthetic procedure.

To be considered biocompatible, a material typically would exhibit the properties of being biologically non-toxic and supporting cell growth and viability. However, the concept of biocompatibility for polymeric coatings utilized in DES has evolved in conjunction with the accumulating clinical data on the use of DES in revascularization procedures. Even though percutaneous coronary interventions have significantly improved the longer-term outcome, introduction of DES has also introduced concerns with regard to vascular inflammation, endothelial dysfunction and late stent thrombosis. The predictors of stent thrombosis associated with DES were attributed to three potential causes: discontinuation of anti-platelet therapy, procedural factors and the DES platform itself (including the stent design, and the potential effect of DES drug and/or polymer). Since the polymer is retained on the stent following the drug release, it has been hypothesized that the chronic presence of polymer can trigger an inflammatory response, contributing to restenosis and thrombosis. Thus, the definition of polymer biocompatibility has to be extended to include the extent of inflammatory effect the polymer may exert on adjacent cells. The mechanism by which polymeric coatings may induce inflammatory response is not well defined.

Monocytes have been proposed to serve as markers, initiators, and promoters of arterial occlusive diseases and monocyte adhesion has been shown to induce local inflammation as well as to promote vascular cell proliferation factors contributing to in-stent restenosis. Furthermore, a vast body of experimental evidence supports the pivotal role of chemokines, such as monocyte chemoattractant protein-1 (MCP1) and interleukins 6 and 8 (IL-6 and IL-8, respectively) in the pathogenesis of vascular disease. In particular, inflammatory responses to arterial injury, which cause continuous recruitment and activation of monocytes mainly through activation of the MCP-1 pathway, play a central role in atherogenesis and restenosis.

The correlation between polymer-induced inflammation and surface hydrophobicity may be useful in designing improved, non-inflammatory, next generation polymers for DES. In addition these data confirms the non-inflammatory makeup of the polymer system design of the present invention.

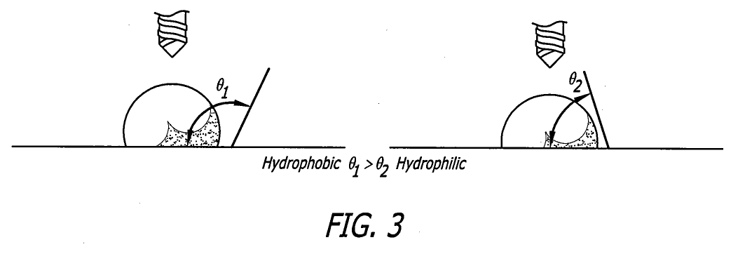

In order to achieve a biocompatible polymer system, a more hydrophilic surface was incorporated. Contact angle measurement is a convenient method to determine relative surface hydrophilicities. A hydrophilic surface allows a drop of water to spread more than a hydrophobic surface as there is less resistance (tension) at the surface. Since the drop is flatter, the angle between the water drop and the surface is smaller than that detected for a hydrophobic surface (FIG. 3). With regards to the polymers C10, C19 and their blends with or without PVP, the data suggests that the C19 polymer with a contact angle of 91° is more hydrophilic than the C10 polymer, with a contact angle of 118°. These results are not unexpected since the C19 polymer contains the hydrophilic vinyl pyrrolidone and vinyl acetate monomers in the polymer architecture. A 70/30 binary blend of C19/C10 and a 63/27/10 ternary blend of C19/C10/PVP, exhibit lower contact angles of 84° and 94°, respectively, indicating their surfaces are more hydrophilic than the C10 polymer. Blending the C10 polymer with the C19 polymer does not show the expected increase in contact angle and in fact the contact angle is somewhat lowered. While it is not surprising that the C19 polymer surface on its own exhibits a lower contact angle compared to the C10 polymer, the surface of a 70/30 blend of C10/C19 does not show a higher contact angle. Instead the contact angle is lower indicating that the surface may be more hydrophilic than the C19 alone. This is due to polymer chain orientation. The C10 and C19 polymers are quite elastomeric, and, as such, the polymer chain segments have mobility at ambient and body temperatures to orient themselves in the most favored conformations. The hydrophobic segments in the C19 polymer orient themselves towards the hydrophobic C10 polymer segments, thereby enhancing surface concentration of hydrophilic segments to exhibit lower contact angles. Addition of 10% PVP on the weight of the blend enhances the contact angle due to the hydrophilic segments reorienting towards the dispersed PVP phase.

The polymer system described herein has a hydrophilic outer surface. Results from examples 7 and 8 demonstrate that the surface of the polymer system is rich in elemental nitrogen suggesting that the vinyl pyrrolidone is present on the surface of the polymer system and hence provides hydrophilic properties.

The surface of the C19 polymer and the surfaces of the blends retain the hydrophilic character contributed by the polar vinyl pyrrolidone units. Furthermore, presence of the hydrophobic C10 polymer in the blends does not lower the concentration of vinyl pyrrolidone units at the surface. These observations are attributed to the orientation of the polar units in the polymer chains in the blends towards the solid-air interface even as the C10 and C19 polymers are highly compatible with each other exhibiting no signs of phase separation.

The polymer system, after self-orienting, has a characteristic hydrophilic surface and hydrophobic core. The hydrophobic core is designed to accommodate a hydrophobic drug selected from the group consisting of anti-proliferatives including, but not limited to, macrolide antibiotics including FKBP-12 binding compounds, estrogens, chaperone inhibitors, protease inhibitors, protein-tyrosine kinase inhibitors, leptomycin B, peroxisome proliferator-activated receptor gamma ligands (PPARγ), hypothemycin, nitric oxide, bisphosphonates, epidermal growth factor inhibitors, antibodies, proteasome inhibitors, antibiotics, anti-inflammatories, anti-sense nucleotides and transforming nucleic acids. Drugs can also refer to bioactive agents including anti-proliferative compounds, cytostatic compounds, toxic compounds, anti-inflammatory compounds, chemotherapeutic agents, analgesics, antibiotics, protease inhibitors, statins, nucleic acids, polypeptides, growth factors and delivery vectors including recombinant micro-organisms, liposomes, and the like.

Exemplary FKBP-12 binding agents include sirolimus (rapamycin) and its derivatives, tacrolimus (FK506), everolimus (certican or RAD-001), temsirolimus (CCI-779 or amorphous rapamycin 42-ester with 3-hydroxy-2-(hydroxymethyl)-2-methylpropionic acid as disclosed in U.S. patent application Ser. No. 10/930,487) and zotarolimus (ABT-578; see U.S. Pat. Nos. 6,015,815 and 6,329,386). Additionally, other rapamycin hydroxyesters as disclosed in U.S. Pat. No. 5,362,718 may be used in combination with the polymers described herein. In addition, paclitaxel and any of its analogs known by those skilled in the art can be incorporated into the hydrophobic core of the self-orienting polymer described herein.

Further, one of the objectives for this polymer system was to ensure the sustained and extended elution of anti-restinosis drugs such as, but not limited to, zotarolimus. Therefore, the drug should be distributed in the polymer uniformly and elute by a diffusion mechanism. Therefore, the polymers should have solubility parameters appropriately matched to each other and to the drug.

In one embodiment, the drug is zotarolimus (Formula 1) (C₅₂H₇₉H₅O₁₂, molecular weight=966.5 g/mol), a tetrazole-containing macrocyclic immunosuppressant. Zotarolimus is an amorphous solid that has extremely low water solubility as demonstrated by very high octanol-water partition coefficient (>4.5 at pH 6.5 and pH 7.4). The mechanism of action of zotarolimus is binding to FKBP12, leading to the formation of a trimeric complex with the protein kinase mTOR (mammalian target of rapamycin) thereby inhibiting its activity. Inhibition of mTOR results in the inhibition of protein phosphorylation events associated with translation of mRNA and cell cycle control.

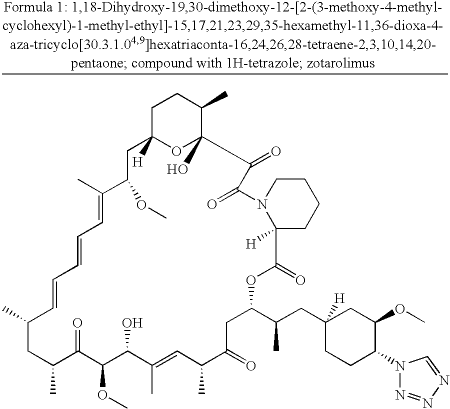

The hydrophobicity of zotarolimus enhances its absorption across the cellular membrane leading to inhibition of neointimal proliferation of the target tissues. Limited water solubility (0.47 μg/ml at pH 6.5 and 0.53 μg/ml at pH 7.4) is highly amenable to the design of a drug-coated stent, and impedes systemic distribution from the stent.

Solubility can be represented as a solubility parameter (δ). The solubility parameter of a molecule is defined as the square root of its cohesive energy density, \sqrt{E_{coh}/V}. E_{coh} is the increase in internal energy per mole of substance if all intermolecular forces are eliminated and is generally estimated from the group contributions to dispersive (δ_{d}), polar (δₚ) and hydrogen bonding (δₕ) forces.

δ=\sqrt{(δ_{d})²+(δ_{d})²+(δₕ)²}

Solubility parameter (δ) values estimated for the C10, C19 polymers and zotarolimus are 17.9 J^{1/2}/cm^{3/2}, 18.0 J^{1/2}/cm^{3/2} and 17.8 J^{1/2}/cm^{3/2} respectively.

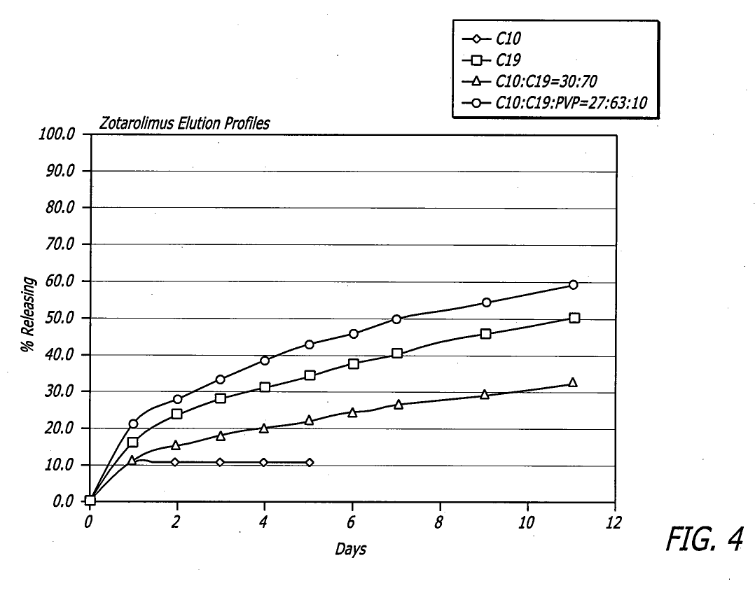

Matching the solubility parameters of the drug and polymer ensures a uniform drug distribution and a sustained release rate, but the amount of drug released at any instant is determined by the free volume in the polymer. Glass transition temperature (T_{g}) is a good indicator of free volume in the polymer. T_{g} is the temperature at which a polymer transitions from a glassy state to a rubbery state. Polymer chains in a rubbery polymer are less tightly entangled and hence offer more free volume than a glassy polymer. A polymer present in a glassy state at or near the body temperature (37° C.) like the C10 polymer (T_{g}=31° C.) elutes only small amounts of a drug while a polymer such as C19 in a rubbery state with more free volume (T_{g}=12° C.) would elute larger amounts (FIG. 4). It is therefore necessary to carefully manipulate the T_{g} of the polymer to achieve the desired elution profile.

One way to achieve the desired amount of drug elution from a polymer coating is to employ a blend of two compatible polymers of appropriate glass transition temperatures. Thus a 30/70 blend of the C10 and C19 polymers formulated at the same drug load offers an intermediate elution profile (FIG. 4).

To achieve the desired elution profile, a third polymer can be added. To complete the polymer system of the present invention, polyvinyl pyrrolidone (PVP) polymer has been added. Addition of 10% of polyvinyl pyrrolidone to the 30/70 C10/C19 blend produces an initial burst and enhances the overall drug release (FIG. 4). The PVP causes the stent coating to swell slightly leading to the increased drug release. Also the PVP chains entangle well with the C10 and C19 polymer chains, and attempts to extract the PVP proved futile. The C19 polymer with 18 mole % vinyl pyrrolidone acts like a compatibilizer for PVP and C10. Absence of a transition for PVP at 177° C. in the differential scanning calorimetry (DSC) scan for the C10/C19/PVP (27/63/10) blend confirms the absence of gross phase separation. It is the non-binding hypothesis that incorporation of PVP further enhances the hydrophilicity of the polymer blend and promotes enhanced biocompatibility relative to other, more hydrophobic, polymers.

It is preferred that when a stent is coated with a drug-containing polymer, the coating is robust, ductile, adheres well to the stent surface and maintains its mechanical integrity as it is tracked through arteries having hard calcified lesions. To meet these requirements, the polymer must have a molecular weight high enough to provide a robust coating. The T_{g} of the coating polymer should also be in the range to offer sufficient flexibility such that the stent coating, upon expansion and deployment, does not crack or show signs of peeling or loss of adhesion. These polymer properties are brought about by a balance of monomers. In one embodiment, vinyl acetate and N-vinyl pyrrolidone monomers offer polarity to the polymers and n-butyl methacrylate and n-hexyl methacrylate monomers provide the flexibility. The polymer blends of the present invention have high enough molecular weights and their blend T_{g} (19.5° C.) is well below body temperature such that they provide a robust, tough, drug-loaded coating that performs satisfactorily when tracked and deployed.

### EXAMPLES

### Example 1

### Synthesis of the C10 Copolymer

The C10 polymer, a copolymer of n-buytl methacrylate and vinyl acetate, was prepared by conventional solution radical polymerization in 1,4-dioxane initiated with 2,2′-azobisisobutyronitrile (AIBN) (Formula 2). The polymerization was carried out at 60° C. to greater than 50% conversion. The synthesis comprised copolymerizing a 60/40 (by weight) mixture of n-butyl methacrylate and vinyl acetate with 0.6% w/w AIBN. The polymer was recovered and purified by five reprecipitations in methyl alcohol. The precipitate was dried in a vacuum oven at 45° C. overnight to constant weight. The use of a 60/40 mixture of n-butyl methacrylate and vinyl acetate yielded a 95%/5% C10 polymer composition.

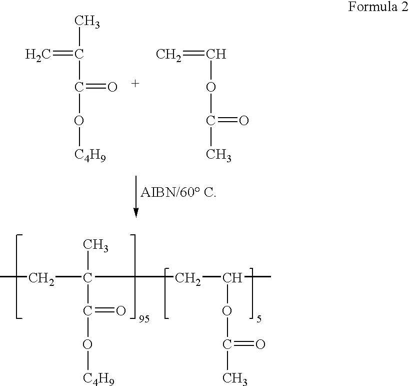

### Example 2

### Synthesis of the C19 Polymer

The C19 polymer, a terpolymer of vinyl acetate, n-hexyl methacrylate and N-vinyl pyrrolidone, was prepared by conventional solution radical polymerization in 1,4-dioxane initiated with AIBN (Formula 3). The polymerization was carried out at 60° C. to greater than 50% conversion. The C19 polymer was prepared by polymerizing a 25%/27%/48% wiw mixture of vinyl acetate, N-vinyl pyrrolidone and n-hexyl methacrylate. The monomers were charged in two stages, the second charge metered to obtain steady-state kinetics. The initiator was 0.825% w/w AIBN. The polymer, being amphiphilic, was purified by cooling the polymer solution in a chloroform/hexanes mixture to −60° C. The precipitate was dried in a vacuum oven at 45° C. overnight to constant weight. The use of a 25%/27%/48% mixture of mixture of vinyl acetate, N-vinyl pyrrolidone and n-hexyl methacrylate yielded a 5%/18%/77% C19 polymer composition.

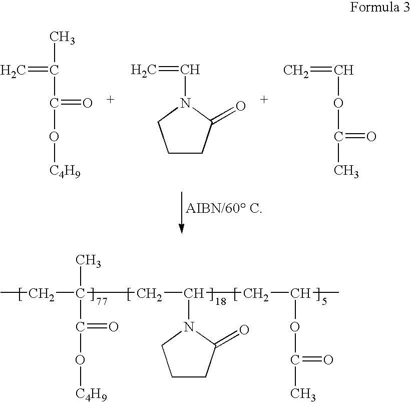

### Example 3

### Synthesis of the C10/C19/PVP Polymer System

In order to synthesize the C10/C19/PVP polymer of the current invention, the C10 copolymer, the C19 terpolymer and a homopolymer of polyvinyl pyrrolidone (PVP) are blended. In one embodiment, the blend comprises 63% C19, 27% C10, and 10% PVP (by weight). This embodiment is sometimes referred to herein as the BioLinx polymer.

### Example 4

### Characterization of Polymers

Polymer molecular weights were measured in tetrahydrofuran (THF) at 35° C. with a Viscotek gel permeation chromatography (GPC) system equipped with a refractive index detector (three columns in series, Polymer Laboratories PLgel (10³, 10⁵ and 10⁶ A⁰) and calibrated with nine polystyrene standards with narrow molecular weights ranging from 4 k to 1750 k. The flow rate was 1 mL/min and injection volume was 100 μL (3 mg/mL concentration).

Polymer compositions were determined from their ¹H nuclear magnetic resonance (NMR) spectra recorded on a Varian, Inc. INOVA-400 MHz NMR spectrometer in CDCl₃. All chemical shifts were relative to tetramethylsilane (TMS). The chemical compositions of polymers were determined by the integrals of proton NMR peaks of individual monomer units.

Polymer glass transition temperatures (T_{g}) were measured on a PerkinElmer, Inc. Diamond Differential Scanning Calorimeter. Samples were scanned twice at 20° C./min from −50° C. to 200° C., and the transitions recorded during the second heat were recorded.

Results of polymer characterization are shown in Table 1. Molecular weights are consistent with the amount of initiator used. Polydispersity indices are typical for radical polymerizations. Glass transition temperatures for C10 and C19 were determined to be 31° C. and 12° C., respectively (FIG. 1), typical of elastomeric polymers. Single glass transition temperatures for these two polymers further confirm their random nature. Any blockiness would have reflected in two or more transitions for the respective blocks.

TABLE 1

| Polymer | Mₙ | M_{w} | PDI | T_{g} (° C.) |
|---|---|---|---|---|
| C10 | 99,992 | 172,278 | 1.72 | 30.5 |
| C19 | 75,387 | 134,300 | 1.78 | 12.9 |
| PVP | 22,550 | 49,975 | 2.22 | 174 |

Mₙ is the number average molecular weight; M_{w} is the weight average molecular weight, PDI is the polydispersity index, and T_{g} is the glass transition temperature.

### Example 5

### Surface Hydrophilicty by Contact Angle Measurement

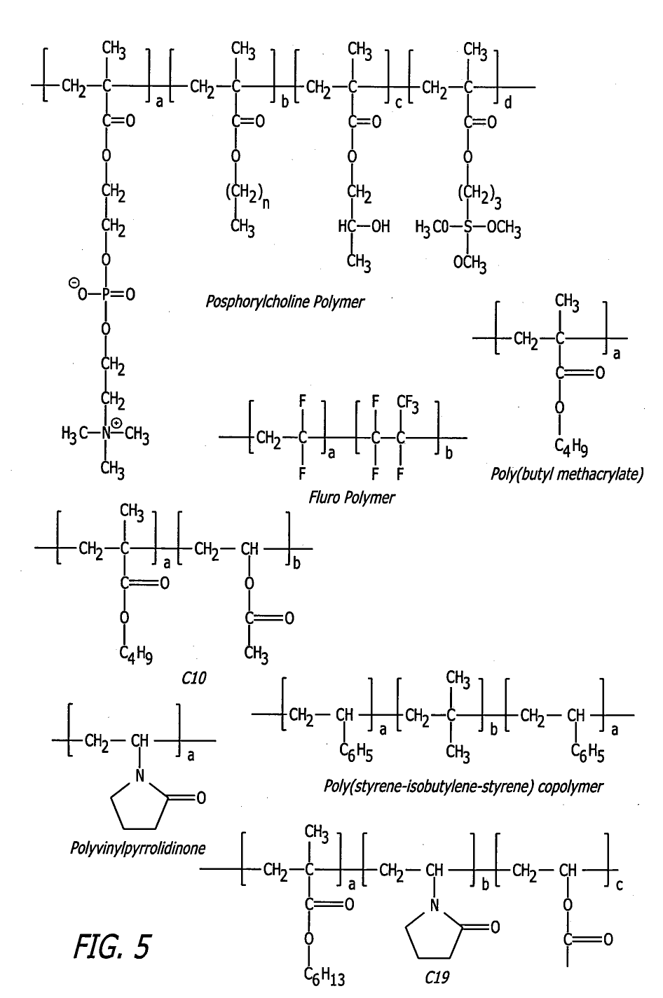

Poly(styrene-isobutylene-styrene) (SIBS) triblock copolymer (grade 073T, 17.5 mol % of styrene, Mw=98 k and PDI=1.28) was supplied by Kaneka Texas Corporation. Poly(butyl methacrylate) (PBMA) and vinylidene fluoride-hexafluoropropylene copolymer (VFH-fluoro polymer) (melting point: 136° C.) were obtained from Sigma-Aldrich. Phosphorylcholine polymer (PC) was provided by Biocompatibles Ltd. Representative chemical structures of these polymers are included in FIG. 5 for reference.

Contact angle measurements were taken with a Goniometer (Rame-Hart Inc.) of flat metal coupons dip-coated with polymer. One drop (10 μL) of deionized water was placed on the polymer surface and illuminated from behind. The image was captured electronically and the angle of contact between the polymer surface and water recorded. Table 2 lists the contact angles for each polymer or polymer blend.

TABLE 2

| Polymer System | Contact Angle | Surface Feature |
|---|---|---|
| Fluoro polymer (VFH) | 129° | Hydrophobic |
| PBMA | 115° | Hydrophobic |
| SIBS | 118° | Hydrophobic |
| C10 | 118° | Hydrophobic |
| C19 | 91° | Hydrophilic |
| C10/C19 | 84° | Hydrophilic |
| C10/C19/PVP | 94° | Hydrophilic |
| PC | 83° | Hydrophilic |

### Example 6

### Raman Spectroscope

Polymer blend compositional uniformity in the polymer-coated stents was determined by confocal Raman microscopy. Raman spectra were acquired using a WiTec confocal Raman microscope equipped with a 785-nm laser source. This laser excitation source was focused using an objective, and the scattered light was collected using a 180° backscatter regime with the laser line intensity being suppressed through the use of an edge filter. The Stokes-shifted Raman scatter was dispersed using 300-grooves/mm grating onto a charge-coupled device. The Raman maps were acquired from regions of the surface of the polymer-coated stents and constructed through a serial mapping process. Confocal depth analysis was performed by acquiring Raman spectra every 250 nm starting from the surface and finishing 5 μm to 10 μm into the stent coating.

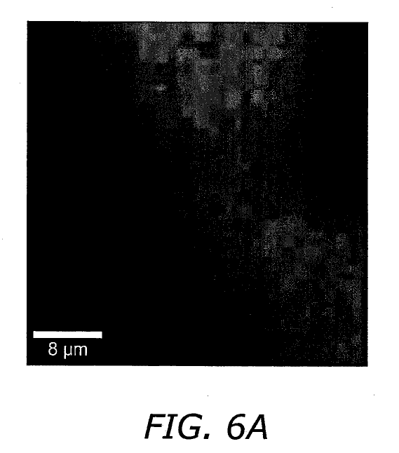

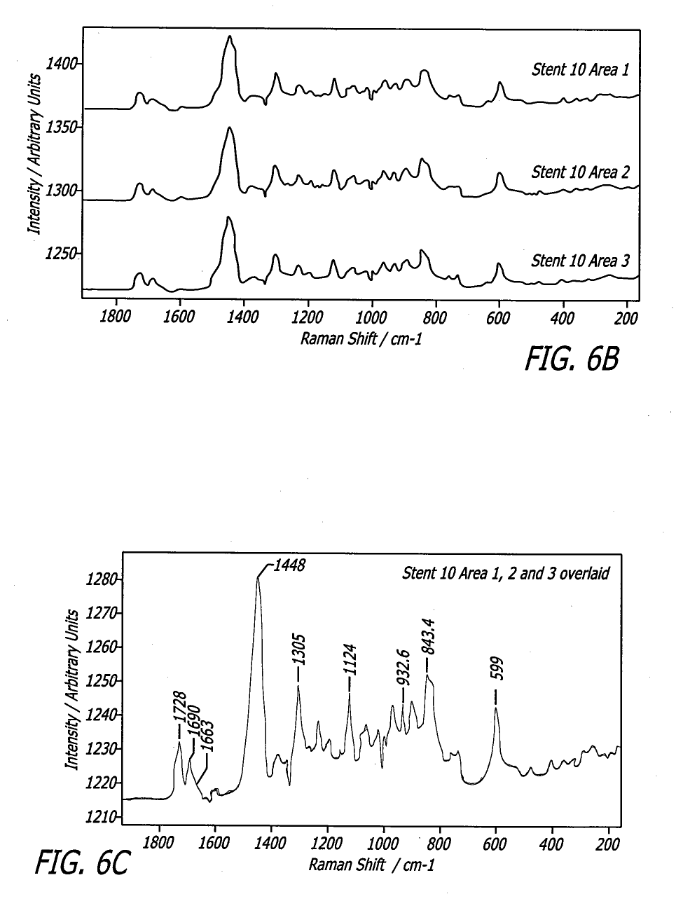

The integral for the 1450 cm⁻¹ peak relating to the asymmetric CH vibration, present in all the polymer components of the polymer stent was acquired to produce the Raman maps. A typical map from a polymer-coated stent within the confocal plane is illustrated in FIG. 6A. Raman spectra were extracted from three 5-μm×5-μm areas of the surface of the polymer-only stent, marked on FIG. 6A. These were compared to assess the chemical composition of the surface of the stent. The spectra acquired from these regions are displayed in FIGS. 6B and 6C. These spectra have been processed to eliminate the interference from an aromatic species (primer coat) also detected in the Raman spectra. The spectra are comparable and can be overlaid (FIG. 6C) suggesting a relatively homogenous mixture of PVP, C19 and C10 polymers at the 5-micron scale.

### Example 7

### Surface Characterization of Polymer System

Samples of C10, C19, C10/C19 (30:70) and C10/C19/PVP polymers were prepared and dip coated onto metal coupons (similar to Example 4). A survey spectrum to determine all elements present (except H) was first acquired from each sample. The spectra were used to obtain quantitative surface composition by integrating the areas under the photoelectron peaks and applying empirical sensitivity factors. The depth of analysis of this technique was on the order of 75 Å. Physical Electronics Quantum 2000 Scanning Electron Spectroscopy for Chemical Analysis (ESCA) equipped with a monochromatic Al Kα x-ray source was employed for the measurement. Other details are as follows:
Analysis area—200 micron spot,
Take-off angle—45°,
Charge correction—C—C, C—H in C1s spectra set to 284.8 eV,
Charge neutralization—Low energy electron and ion floods.
Results are displayed in Table 3 below.

TABLE 3

| Polymer System | % N | % C | % O |
|---|---|---|---|
| C10 | 0 | 79.7 | 20.3 |
| C19 | 1.6 | 81.5 | 16.9 |
| C10/C19 (30:70) | 1.4 | 81.4 | 17.2 |
| C10/C19/PVP (27:63:10) | 1.3 | 81.2 | 17.5 |

It is apparent from the table that percent elemental nitrogen for the C19 polymer corresponds with the vinyl pyrrolidone content in the polymer. Presence of the C10 polymer (with no vinyl pyrrolidone in its architecture) in the 70/30 C19/C10 blend or the C10/C19/PVP polymer system does not, however, show a corresponding drop in percent nitrogen. These results suggest that the surfaces of the binary and ternary blends substantially retain vinyl pyrrolidone moieties at the surface and confer the hydrophilic character.

### Example 8

### Surface Chemical Composition by Attenuated Total Reflectance

Metal coupons coated with C10, C19, PVP, and C10/C19/PVP, and were prepared similar to the examples above. Infrared spectra were acquired using a Nicolet Avatar spectrometer with a Centaurus ATR microscope, equipped with a germanium crystal. Data were acquired at a resolution of 4 cm⁻¹ added over 128 scans. The analysis spot was approximately 30 μm×30 μm. Using a germanium crystal, approximately the top 0.5 μm is analyzed.

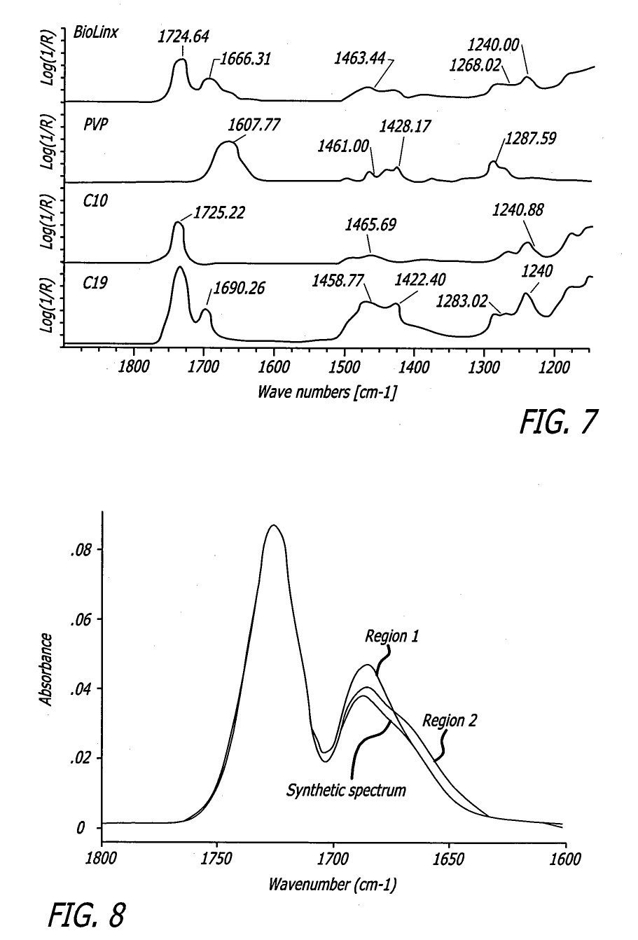

Attenuated total reflectance (ATR) spectra (4000 cm⁻¹ to 650 cm⁻¹) for the C10, C19, PVP and C10/C19/PVP polymers have surfaces shown in FIG. 7. The ester carbonyls of methacrylate and acetate units appear at 1725 cm⁻¹. The pyrrolidone amide gives a carbonyl peak at 1670 cm⁻¹ in pure PVP. However the same is shifted to 1690 cm⁻¹ in copolymers. Such a shift is interpreted as a break-up of self-associated amide carbonyls in the presence of ester carbonyls. Several different regions along the polymer surfaces were compared and FIG. 8 shows an expansion around the carbonyl region (1800-1600 cm⁻¹) scaled to the ester peak at 1725 cm⁻¹. The pyrrolidone amide peak shows differences with the main peak at 1685 cm⁻¹ (pyrrolidone within the C19 polymer environment) but in some instances with a shoulder at 1670 cm⁻¹. This was investigated further by composing a spectrum synthesized digitally by adding spectra of C10, C19 and PVP with appropriate scaling factors (C19=0.63, C10=0.27 and PVP=0.1), A comparison of the spectra for the polymer system in the expanded carbonyl region (1800-1600 cm⁻¹) to the synthetic spectrum clearly demonstrates that the vinyl pyrrolidone content at the surface is disproportionately higher. Other reasons for such a behavior like the presence of water leading to different hydrogen bonding patterns were also investigated. Inspection of O—H stretch region of the different areas on the surface upon analysis suggested that the water content is low and at similar levels.

### Example 9

### Coating a Stent with a Polymer and a Drug

Stents were coated with a drug and polymer. Zotarolimus and polymers were weighed (35/65 weight ratio) into the same vial. Chloroform was pipetted into the vial to obtain a 1% concentration of the drug-polymer mixture. The solution was filtered with 0.2-μm polytetrafluoroethylene (PTFE) filter into another clean vial, ready for coating. The solution of polymer blend with the drug was sprayed onto parylene C-primed Driver® stents using ultrasonic spray equipment. Dried, coated stents were mounted on balloon catheters and sterilized with ethylene oxide.

Durability of the coated stents was determined by tracking through a simulated lesion, expanding at nominal (9 atm) pressure and then inspecting at 40× using optical microscopy. Post-tracked stents were also inspected by scanning electron microscopy for signs of delamination, cracking and excessive wear.

### Example 10

### In Vitro Drug Elution

Coated stents were placed in 2 mL of 10 mM trishydoxymethylaminomethane (pH 6.5) buffer containing 0.4% sodium dodecyl sulfate (TRIS-SDS buffer) and incubated at 37° C. Samples were taken every 24 hours up to 7 days. Fresh TRIS-SDS buffer was used at each time point. After 7 days, the samples were taken every 48 hours. Test samples were analyzed for drug concentration using high-performance liquid chromatography (HPLC).

Studies of cumulative elution over a 28 day period show that an initial burst of drug is released (FIG. 4) from the drug-polymer complex over the first 48 hours, which slows asymptotically to a sustained release rate over subsequent weeks. Such a release profile was desired to provide both early and late sustained control of neointimal responses.

### Example 11

### Polymer Meets Guidelines of Established Standards

Polymer coated stents were tested to ensure safety and biocompatibility as per guidelines of the American National Standards Institute (ANSI), the Association for the Advancement of Medical Instrumentation (AAMI) and the International Organization for Standardization (ISO). More specifically, compliance with ANSI/AAMI/ISO 10993-1 and G95-1 was evaluated.

The test article, when subjected to an in vitro cytotoxicity study to determine if leachables from the test extract would cause cytotoxicity, showed no signs of causing cell lysis or toxicity (Grade 0), and the positive, negative and reagent controls performed as anticipated. The mean hemolytic index for the test article was 0% in the in vitro hemolysis test performed on any leachable chemicals from the test article. As such the polymer system is nonhemolytic. The controls performed as anticipated.

USP and ISO Acute Systemic Toxicity (in the mouse) were performed to determine whether leachables extracted from the material would cause acute systemic toxicity following injection into mice. The test article was extracted in both 0.9% sodium chloride USP (SC) and sesame oil, NF (SO). Single doses of the test article extract were injected into each of five mice per extract by either the intravenous (SC extract) or intraperitoneal (SO extract) route. The control mice were similarly dosed. The animals were observed immediately and at 4, 24, 48 and 72 hours postsystemic injection. There was no mortality or systemic toxicity in either; the test animals responded similarly to the controls.

ISO Acute Intracutaneous Reactivity when conducted in rabbits with extracts as above by injecting intracutaneously did not produce any erythema/edema. ISO Sensitization testing done on guinea pigs with extracts also proved negative.

### Example 12

### Monocyte Adhesion Experiments

A polymer solution was prepared by dissolving 400 mg of polymer in 100 mL (4 mg/mL) of an appropriate high purity high performance liquid chromatography (HPLC)/Biotech grade solvent. Dichloromethane was the preferred solvent for C10, C19, poly(butyl methacrylate) (PBMA), poly(styrene-isobutylene-styrene) (SIBS), C10/C19/PVP and tissue culture polystyrene (TCPS). Phosphorylcholine (PC) and the fluoropolymer were dissolved in ethanol and 3:1 acetone:cyclohexanone respectively. The solution was filtered with a 0.45 um polytetrafluroethylene (PTFE) filter and 220 uL was dispensed into 96 well cell culture plates. The solvent was evaporated inside a fume hood for twelve hours, followed by treatment under high vacuum (<1 mmHg) at room temperature overnight. The PC polymer was dried at 70° C. for 4.5 hours (no vacuum). The TCPS was dried at 105° C. and the fluoropolymer was dried at 135° C.

Monocytic U937 cells were purchased from ATCC Cell Biology Collection and maintained in culture according to vendor recommendations. U937 cells were seeded at 1×10⁵/well onto polymer coated 96 well plates. The cells were stimulated with lipopolysaccharide (LPS) (100 ng/mL) and phorbol-12-myristate-13-acetate (PMA) (100 ng/mL) to induce differentiation and inflammatory activation. Activated monocytes were then incubated on polymer for 24 hours at 37° C. Adhesion of U937 cells to the polymer scaffold was assessed by calcein uptake as detailed in the following protocol. U937 cells were fluorescently labeled by incubation with calcein (1 mg/mL) for 30 minutes. Calcein is a fluorescent dye hydrolyzed by esterases in viable cells. The percent uptake of calcein is directly proportional to the number of viable cells in the well. The percent of cell adhesion was determined by reading the fluorescent measurements prior to and after gentle PBS washing of the adherent monocytes from the polymer coated plates. U937 cells stimulated with LPS+PMA that were seeded on top of tissue culture polystyrene coated wells (TCPS) served as a positive control and the unstimulated U937 cells served as a negative control. To calculate the relative percent adhesion, the ratio between the fluorescent measurement prior to and after gentle PBS washing of the adherent monocytes from the polymer coated plates was first determined. This ratio was then normalized against the ratio obtained for the positive control and multiplied by a factor of 100 for the relative percent adhesion.

In order to evaluate, in vitro, the polymers potential to elicit an inflammatory response, the adhesion of activated monocytes to the components of various polymers were measured, individually and in combination (C10, C19, and C10/C19).

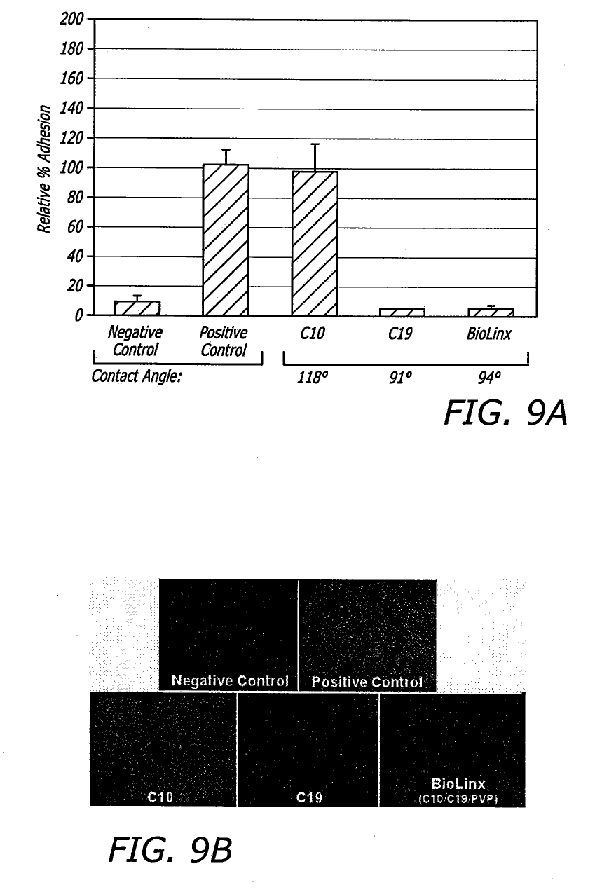

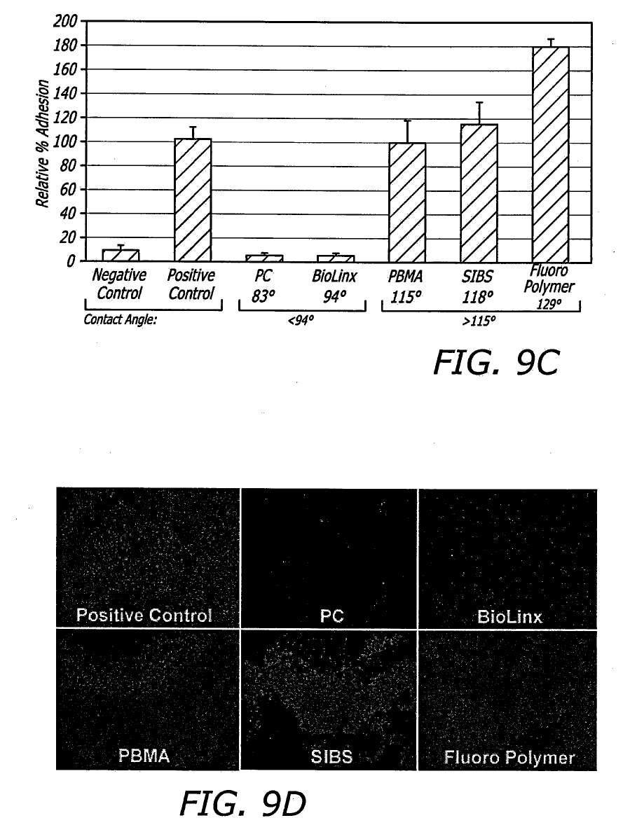

Activated monocytic cells were placed on top of the various polymer-coated wells and the level of adherence was evaluated. As anticipated, monocytic cell adherence was substantially induced upon the inflammatory activation with LPS and PMA (13 fold difference positive vs. negative controls, FIG. 9). In a similar manner, a robust adherence of the stimulated monocytes to the hydrophobic polymer C10 was observed (12.5 fold induction over non-stimulated, negative control cells). In contrast, the adherence of stimulated monocytes to polymers containing the hydrophilic component C19 (C19 and C10/C19/PVP) was minimal and did not differ significantly from the unstimulated control cells (FIG. 9A and B). The correlation between the relative hydrophobicity and the adherence of activated monocytes is further substantiated by the results shown in FIG. 9C and D; While the monocytic adherence to the relatively hydrophilic polymers (contact angle<94°), PC and the C10/C19/PVP polymer system was low, it was considerably enhanced with regards to the more hydrophobic polymers (contact angle>115°), PBMA, SIBS and VFH-flouro polymer (12.6, 15 and 23 fold induction over non-stimulated, negative control cells, respectively).

### Example 13

### Porcine Studies

A series of in vivo studies were conducted in the porcine coronary artery model over time periods ranging up to 180 days. Polymer coated stents with and without drug were implanted in 72 juvenile domestic farm swines and 91 Yucatan mini-swines. The National Institute of Health (NIH) guidelines for the care and use of lab animals were strictly observed. The animals were pre-medicated with aspirin 650 mg and clopidogrel 150 mg 12-24 hours before the stenting procedure. Angiographic images of the vessels were obtained to identify the target location for stent deployment. A visual estimate of vessel diameter was completed in order to identify a target vessel and location suitable for the stents with a balloon to artery ratio of approximately 1.1-1.2 to 1. Stent implantation was completed in two or three coronary arteries (right coronary artery (RCA), left anterior descending artery (LAD) and/or left circumflex artery (LCX)) per animal, depending on the suitability of the anatomy. Quantitative analysis of coronary angiograms was completed off-line with the Medis, Inc. analysis software. The animals were treated with aspirin 81 mg and clopidogrel 75 mg daily by mouth for the duration of the in-life phase (clopidogrel administered out to 28 days for 90 and 180 day studies). At 7, 28, 90 and 180 days, the animals underwent follow-up angiographic procedures. After completion of angiography, the animals were euthanized with an overdose of sodium pentobarbital.

A comparison of arterial tissue inflammation scores 180 days following implantation revealed very little or mild inflammatory responses, which were not significantly different between groups, for both the bare metal and polymer-only coated stents (score of less than 1 for both groups). A 28-day polymer-safety study demonstrated a similar result, with the bare metal and polymer-coated stents producing inflammation scores that were not significantly different.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used herein (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate and do not pose a limitation on the scope otherwise claimed. No language in the specification should be construed as indicating that any non-claimed element is essential to the embodiments disclosed herein.

Groupings of alternative elements or embodiments disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments are described herein, including the best mode, if known to the inventors at the time of filing. Of course, variations on these described embodiments will become apparent to those of ordinary skill in the art upon reading the foregoing description. The inventor expects skilled artisans to employ such variations as appropriate. Practice of modifications and equivalents of the subject matter recited in the claims is expected. Moreover, any combination of the above-described elements in all possible variations thereof is encompassed herein unless otherwise indicated or otherwise clearly contradicted by context.

Furthermore, references have been made to patents and printed publications throughout this specification. Each of the above-cited references and printed publications individually are incorporated herein by reference in their entirety.

In closing, it is to be understood that the embodiments disclosed herein are for illustrative purposes. Other modifications may be employed and are within the scope of the claims. Thus, by way of example, but not of limitation, alternative configurations may be utilized in accordance with the teachings herein. Accordingly, the teachings herein are not limited to that precisely as shown and described.

I claim:

1. A polymer system for coating medical devices comprising:
a polymer blend, wherein said polymer blend forms a self-orienting polymer coating having an outer surface, and wherein said polymer coating has hydrophilic groups oriented towards said outer surface.

2. The polymer system of claim 1 wherein said polymer blend comprises at least one of a homopolymer, a copolymer or a terpolymer.

3. The polymer system of claim 1 wherein said self-orienting polymer coating is capable of controlled release of a hydrophobic drug.

4. The polymer system of claim 3 wherein said self-orienting polymer coating is biocompatible.

5. The polymer system according to claim 3 wherein said hydrophobic drug is selected from the group consisting of anti-proliferatives, estrogens, chaperone inhibitors, protease inhibitors, protein-tyrosine kinase inhibitors, leptomycin B, peroxisome proliferator-activated receptor gamma ligands (PPARγ), hypothemycin, nitric oxide, bisphosphonates, epidermal growth factor inhibitors, antibodies, proteasome inhibitors, antibiotics, anti-inflammatories, anti-sense nucleotides and transforming nucleic acids.

6. The medical device according to claim 3 wherein said drug comprises at least one compound selected from the group consisting of sirolimus (rapamycin) and its analogs, tacrolimus (FK506), everolimus (certican), temsirolimus (CCI-779), zotarolimus (ABT-578), paclitaxel and its analogs.

7. The polymer system of claim 1 wherein said outer surface has a water contact angle of <95°.

8. The polymer system according to claim 2 wherein said copolymer comprises alkyl methacrylate monomers, vinyl acetate monomers or combinations thereof.

9. The polymer system according to claim 2 wherein said terpolymer comprises alkyl methacrylate monomers, vinyl pyrrolidone monomers, vinyl acetate monomers or combinations thereof.

10. The polymer system according to claim 2 wherein said homopolymer comprises vinyl pyrrolidone monomers.

11. The polymer system according to claim 1, wherein said coating's outer surface has a Hilderbrand solubility parameter of about 15 to about 20.

12. The polymer system according to claim 2 comprising a ratio of terpolymer to copolymer to homopolymer, wherein said ratio is from about 40:40:20 to about 88:10:2.

13. A biocompatible medical device comprising:
a substrate comprising a coating on a surface;
wherein said coating comprises a self-orienting polymer system,
further wherein said polymer system comprises an outer surface; and
further wherein said polymer system has hydrophilic groups oriented towards said outer surface.

14. The medical device according to claim 13 wherein said polymer system comprises a at least one of a homopolymer, a copolymer, or a terpolymer.

15. The medical device according to claim 13 wherein said medical device is implantable and is selected from the group consisting of heart valves, stents, pacemaker leads and combinations thereof.

16. The medical device according to claim 13 wherein said self-orienting polymer coating is capable of controlled release of a hydrophobic drug.

17. The medical device according to claim 16 wherein said hydrophobic drug comprises at least one compound selected from the group consisting of sirolimus (rapamycin) and its analogs, tacrolimus (FK506), everolimus (certican), temsirolimus (CCI-779), zotarolimus (ABT-578), paclitaxel and its analogs.

18. The medical device according to claim 14 wherein said copolymer comprises alkyl methacrylate monomers, vinyl acetate monomers, or combinations thereof.

19. The medical device according to claim 14 wherein said terpolymer comprises alkyll methacrylate monomers, vinyl pyrrolidone monomers, vinyl acetate monomers, or combinations thereof.

20. The medical device according to claim 14 wherein said homopolymer comprises vinyl pyrrolidone monomers.

21. The medical device according to claim 13 wherein said outer surface has a water contact angle of <95°.

22. The medical device according to claim 13, wherein said outer surface has a Hilderbrand solubility parameter of about 15 to about 20.

23. The polymer system according to claim 13 comprising a ratio of terpolymer to copolymer to homopolymer, wherein said ratio is from about 40:40:20 to about 88:10:2.

24. A biocompatible implantable stent comprising:
a self-orienting polymer system coating comprising at least one of polyvinylpyrrolidone, alkyl methacrylate, vinyl acetate, and vinylpyrrolidone;
wherein said polymer system further comprises a hydrophilic surface and a hydrophobic core;
wherein said hydrophobic core comprises a hydrophobic drug and can provide controlled release;
wherein said drug comprises zotarolimus.
